(19) United States
(12) Patent Application Publication
(10) Pub. No.: US 2024/0390639 A1
(43) Pub. Date: Nov. 28, 2024

(54) INTRAVASCULAR DEVICE WITH NESTED HYPOTUBE CONFIGURATION

(71) Applicant: SCIENTIA VASCULAR, INC., West Valley City, UT (US)

(72) Inventors: Nolan LESUEUR, West Valley City, UT (US); William Rigby ECCLES, Centerville, UT (US); Edward J. SNYDER, Park City, UT (US); Brandon Rodney POPE, Salt Lake City, UT (US)

(21) Appl. No.: 18/667,535

(22) Filed: May 17, 2024

Related U.S. Application Data
(60) Provisional application No. 63/468,211, filed on May 22, 2023.

(51) Int. Cl. A61M 25/00; A61M 25/09

(52) U.S. Cl. CPC A61M 25/0045; A61M 2025/0004; A61M 25/09

(57) ABSTRACT

Disclosed are intravascular devices for accommodating large-bore catheters while maintaining good flexibility of the device. The intravascular device includes a plurality of hypotubes arranged in a nested configuration. The intravascular device provides a relatively large overall size while minimizing increases in lateral bending stiffness.

### CROSS-REFERENCE TO RELATED APPLICATIONS

This application claims priority to and the benefit of U.S. Provisional Patent Application No. 63/468,211, filed on May 22, 2023 and titled “Nested Microfabricated Hypotubes in Large Diameter Guidewire,” the entirety of which is incorporated herein by reference.

### BACKGROUND

Guidewire devices are often used to lead or guide catheters or other interventional devices to a targeted anatomical location within a patient's body. Typically, guidewires are passed into and through a patient's vasculature in order to reach the target location, which may be at or near the patient's heart or brain, for example. Radiographic imaging is typically utilized to assist in navigating a guidewire to the targeted location. In many instances, a guidewire is placed within the body during the interventional procedure where it can be used to guide multiple catheters or other interventional devices to the targeted anatomical location.

Guidewires are available with various outer diameter sizes. Widely utilized sizes include 0.010, 0.014, 0.016, 0.018, 0.024, 0.035, and 0.038 inches, for example, though they may also be smaller or larger in diameter. Because torque transmission is a function of diameter, larger diameter guidewires typically have greater torque transmission (the ability to effectively transfer torque from proximal portions of the wire to more distal portions of the wire). On the other hand, smaller diameter guidewires typically have greater flexibility.

A catheter used in conjunction with a guidewire will be sized with an inner diameter (the diameter of the bore of the catheter) somewhat larger than the outer diameter of the guidewire to enable the catheter to be positioned over and translated upon the guidewire. The difference in size between the guidewire and catheter can affect the ability of the catheter to travel along the guidewire. For example, the larger the annular space between the outer diameter of the guidewire and the inner diameter of the catheter, the greater the amount of radial play the catheter may experience and the more difficult it may be to navigate the catheter over the guidewire. With excessive radial play, the distal end of the catheter may have a higher risk of catching against vasculature or other anatomy of the patient rather than smoothly following along the guidewire path.

Often, a guidewire size is selected to minimize the amount of annular space between the guidewire and a catheter with a given size required or desired for a particular procedure, to thereby limit the types of issues described above. However, guidewires suffer from excessive stiffness when sized with a sufficiently large diameter to accommodate large-bore catheters. Instead, to form an intravascular device with a large diameter suitable for guiding a large-bore catheter, it is common practice to first direct a small guidewire through a patient's vasculature and then thread a microcatheter over the guidewire, followed by an intermediate catheter and then the large-bore catheter. However, this practice increases the time needed to deliver the distal end of the large-bore catheter to the targeted anatomical location and increases the operational complexity of the procedure.

What is needed, therefore, is a guidewire device capable of being manufactured with a relatively large outer diameter, that minimizes the annular space between the guidewire and large-bore catheters, and that is also capable of providing sufficient flexibility and torquability. A large-bore catheter would more easily travel along such a guidewire, decreasing surgical complications, potential injury, and operation time.

### SUMMARY

Disclosed are intravascular devices comprising an inner member (e.g., a solid core wire or simply “core”) having a proximal section and a distal section, and a plurality of hypotubes. Each hypotube of the plurality of hypotubes includes a proximal section and a distal section. At least a portion of the inner member passes into and is encompassed by the plurality of hypotubes. The hypotubes are arranged in a nested configuration such that a portion of each hypotube (except for the outermost hypotube) passes into and is encompassed by at least one other hypotube.

The disclosed devices can provide a guidewire with a relatively large outer diameter while minimizing increases in lateral bending stiffness that usually accompany larger diameter guidewires. This can enable more effective use of large-bore catheters. That is, a guidewire device as disclosed herein can minimize the annular space between the guidewire and a large-bore catheter, while also enabling effective flexibility and torquability relative to conventional guidewires of similar size. A large-bore catheter can more easily travel along a guidewire as disclosed herein, decreasing surgical complications, potential injury, and operation time.

In some embodiments, the intravascular device comprises at least two hypotubes or at least three hypotubes. In some embodiments, except for the outermost hypotube, a proximal end of each hypotube is encompassed by at least one other hypotube.

In some embodiments, starting at a distal end of the device, an outer diameter of the device increases along a proximal direction at discrete points coinciding with transitions from one hypotube to another. For example, the outer diameter of the device increases by 0.005 inches to 0.020 inches, or by 0.010 inches to 0.015 inches, at each transition from one hypotube to another.

In some embodiments, the proximal end of the inner member is encompassed by at least one hypotube, such as by each of the plurality of hypotubes. In some embodiments, a proximal end of the inner member and proximal ends of one or more hypotubes are coincident at a proximal end of the intravascular device. Alternatively, the proximal end of the inner member can extend farther proximally than the plurality of hypotubes.

In some embodiments, one or more hypotubes of the plurality of hypotubes are formed from nitinol. In some embodiments, the inner member is a solid core wire, such as one formed from stainless steel.

In some embodiments, the plurality of hypotubes include a plurality of fenestrations. For example, the fenestrations can form an arrangement of circumferentially extending rings connected by axially extending beams.

In some embodiments, the plurality of hypotubes defines a hypotube coverage length, and the outermost hypotube has a length that is more than 50% of the hypotube coverage length.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an embodiment of a guidewire device having a core and an outer tube and which may utilize one or more of the components described herein;

FIG. 2 illustrates an exemplary embodiment of a guidewire device and a large-bore catheter traveling along the guidewire device, the catheter displaying large leading shoulders.

FIGS. 3A through 3D illustrate exemplary guidewire devices including a plurality of nested hypotubes.

FIG. 4 illustrates an exemplary embodiment of a guidewire device including a plurality of nested hypotubes from which are cut a plurality of fenestrations.

### DETAILED DESCRIPTION

### Example Guidewire Structural Features

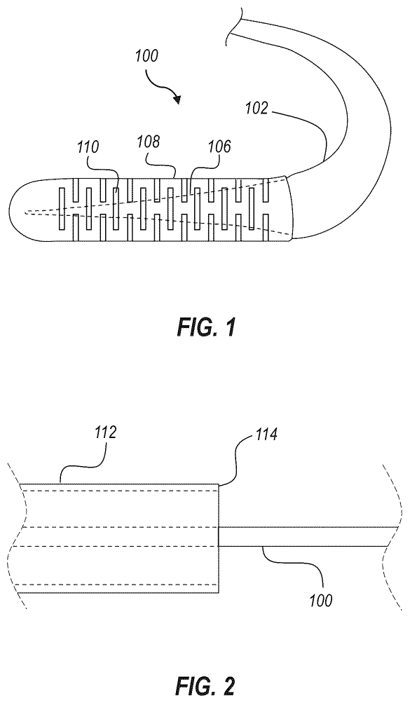

FIG. 1 schematically illustrates the general components of a guidewire 100 that may utilize one or more features described herein. The illustrated guidewire 100 includes an inner member (core 102, in this embodiment) and an outer tube 108. The core 102 includes a distal section 106 that extends into the outer tube 108 as shown. The distal section 106 may be tapered, either continuously or in one or more discrete sections, so that the more distal sections have a smaller diameter and greater flexibility than more proximal sections. In some embodiments, the most distal section of the core 102 may be flattened into a ribbon-like shape with a flat, rectangular, or oblong cross section. For example, the distal section 106 may be ground so as to progressively taper to a smaller diameter at the distal end.

The core 102 and the tube 108 are typically formed from different materials. For example, the tube 108 is preferably formed from a relatively elastic material such as nitinol, whereas the core 102 may be formed from a relatively less elastic material such as stainless steel. Forming the core 102 from stainless steel may be advantageous because it allows the distal tip to hold a shape when selectively bent/shaped by an operator and because stainless steel provides is readily able to plastically deform and hold a shape. While these materials are presently preferred, other suitable materials such as polymers or other metals/alloys may also be utilized for the core 102 and/or tube 108.

The tube 108 is coupled to the core 102 (e.g., using adhesive, soldering, and/or welding) in a manner that beneficially allows torsional forces to be transmitted from the core 102 to the tube 108 and thereby to be further transmitted distally by the tube 108. A medical grade adhesive or other suitable material may be used to couple the tube 108 to the core wire 102 at the distal end of the device to form an atraumatic covering.

The outer tube 108 may include a cut pattern that forms fenestrations 110 in the tube. The pattern of fenestrations 110 can include axially extending “beams” and circumferentially extending “rings” as shown, and/or may be arranged to provide desired flexibility characteristics to the tube 108, including the promotion of preferred bending directions, the reduction or elimination of preferred bending directions, or gradient increases in flexibility along the longitudinal axis, for example. Examples of cut patterns and other guidewire device features that may be utilized in the guidewire devices described herein are provided in detail in U.S. Pat. Nos. 10,821,268, 11,052,228, and in 11,369,351, the entireties of each of which are incorporated herein by this reference.

Suitable cut patterns include, for example, a three-beam cut pattern, two-beam cut pattern, and one-beam cut pattern. A “two-beam” cut pattern or section refers to a section of the outer tube comprising two axially extending “beams” between each pair of consecutive circumferentially extending “rings,” a “one-beam” cut pattern or section refers to a section of the outer tube comprising a single axially extending “beam” between each pair of consecutive circumferentially extending “rings,” and so on.

The beams and rings may be arranged along the length of a given section of the tube to form various cut patterns, including a linear pattern, helical pattern, or a distributed pattern that is non-helical and non-linear (see U.S. Pat. No. 11,369,351).

The proximal section of the guidewire device 100 (the portion extending proximally from the tube 108) extends proximally to a length necessary to provide sufficient guidewire length for delivery to a targeted anatomical area. The guidewire device 100 typically has a length ranging from about 50 cm to about 350 cm, more commonly about 200 cm, depending on particular application needs. The tube 108 may have a length ranging from about 5 cm to about 65 cm, more typically about 15 cm to about 50 cm, such as about 25 cm to about 40 cm.

The guidewire device 100 may have a diameter of about 0.010 inches to about 0.038 inches, though larger or smaller sizes may also be utilized depending on particular application needs. For example, particular embodiments may have outer diameter sizes corresponding to standard guidewire sizes such as 0.014 inches, 0.016 inches, 0.018 inches, 0.024 inches, 0.035 inches, 0.038 inches, or other such sizes common to guidewire devices. The distal section 106 of the core 102 may taper to a diameter of about 0.002 inches, or a diameter within a range of about 0.001 to 0.005 inches. In some embodiments, the distal tip may be flattened (e.g., to a rectangular cross section) to further enhance bending flexibility while minimizing reductions in cross-sectional area needed for tensile strength. In such embodiments, the cross section may have dimensions of about 0.001 inches by 0.003 inches, for example. In some embodiments, the tube 108 has a length within a range of about 3 to 100 cm.

Additional features and details regarding the foregoing components are described in further detail below. The following examples may be particularly beneficial in applications where the corresponding catheter is about sized at 0.027 inches or greater, and the guidewire is thus beneficially sized at about 0.024 inches or greater in order to limit the amount of annular space between the inner surface of the catheter and the outer surface of the guidewire. In such implementations, the guidewires described herein are able to provide sufficient diameter in the distal sections of the device to minimize the annular space between the guidewire and a corresponding catheter, while still maintaining effective flexibility. These sizes are not limiting, however, and the same features and details described below may also be utilized in guidewires that are smaller or larger than 0.024 inches.

### Guidewire and Catheter Relative Sizing

A guidewire device may be used to direct a distal end of the catheter to a targeted anatomical location. After the distal end of the guidewire is delivered to the target, a catheter is placed onto the proximal section of the guidewire. The catheter is then pushed along the guidewire until the distal end of the catheter reaches the target anatomy.

The outer diameter of the guidewire will be somewhat smaller than the inner diameter of the catheter, resulting in an annular space between the guidewire and the catheter. Larger differences between the outer diameter of the guidewire and the inner diameter of the catheter creates excessive annular space, resulting in greater radial play of the catheter as it travels along the guidewire. Large amounts of radial play can cause the distal end of the catheter to deviate from the path of the guidewire and thus impede the smooth translation of the catheter along the guidewire.

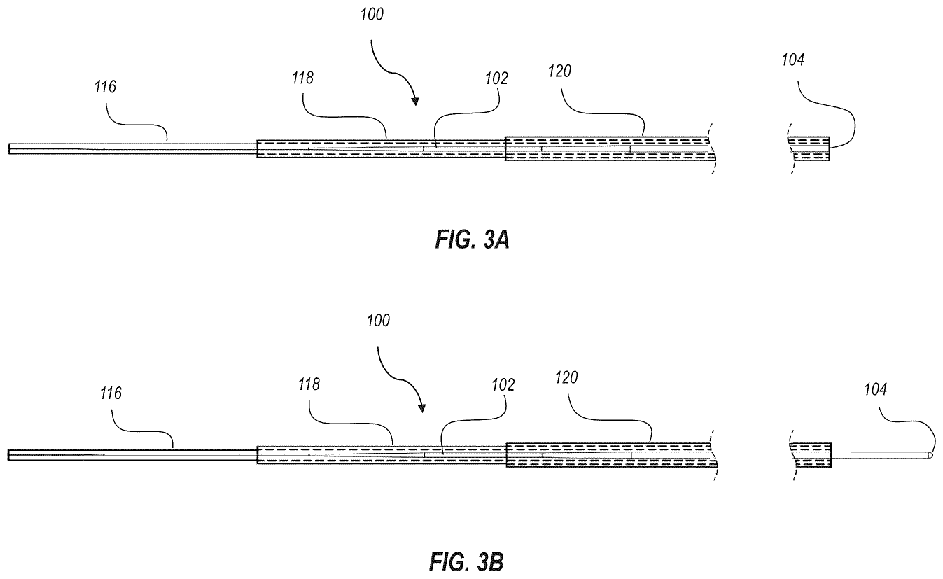

FIG. 2 illustrates a catheter 112 traveling along the guidewire device 100. The outer diameter of the guidewire device 100 depicted in FIG. 2 has a substantially smaller outer diameter than the inner diameter of the catheter 112. The catheter 112 will therefore exhibit large leading shoulders 114 located at the distal end of the catheter, as shown in FIG. 2. Such leading shoulders 114 are formed when there is a substantial difference between the outer diameter of the guidewire 100 and the inner diameter of the catheter 112. Large leading shoulders 114 may catch on intravascular tissue as the catheter 112 travels along the guidewire device 100 and thus hinder the smooth navigation of the catheter to the target. The catheter 112 is also more likely to catch or bind against the guidewire 100 when there is excessive annular space between the outer surfaces of the guidewire 100 and the inner surfaces of the catheter 112.

A large-bore catheter may be needed for certain medical procedures, and thus a large diameter guidewire would be beneficial for such applications. However, the bending flexibility of a guidewire device typically decreases as the diameter of the core increases, diminishing the navigable case of the device through a patient's vasculature. Because of this, in a procedure that calls for a large-bore catheter, the conventional approach utilizes a relatively small guidewire and multiple levels of increasingly larger catheters. For example, in a common approach, a relatively small-diameter guidewire is first navigated to the targeted anatomical location, then a microcatheter is passed over the guidewire device, then an intermediate catheter is passed over the microcatheter. Finally, the large-bore catheter is translated over the intermediate catheter.

However, the need to pass additional catheters such as a microcatheter and an intermediate catheter over the guidewire device increases the overall operation time and risk of complication of a medical procedure. Instead of the conventional approach, when attempting to use a large-bore catheter, a guidewire including a relatively large outer diameter while still maintaining sufficient flexibility would be preferred.

### Example Large Diameter Devices

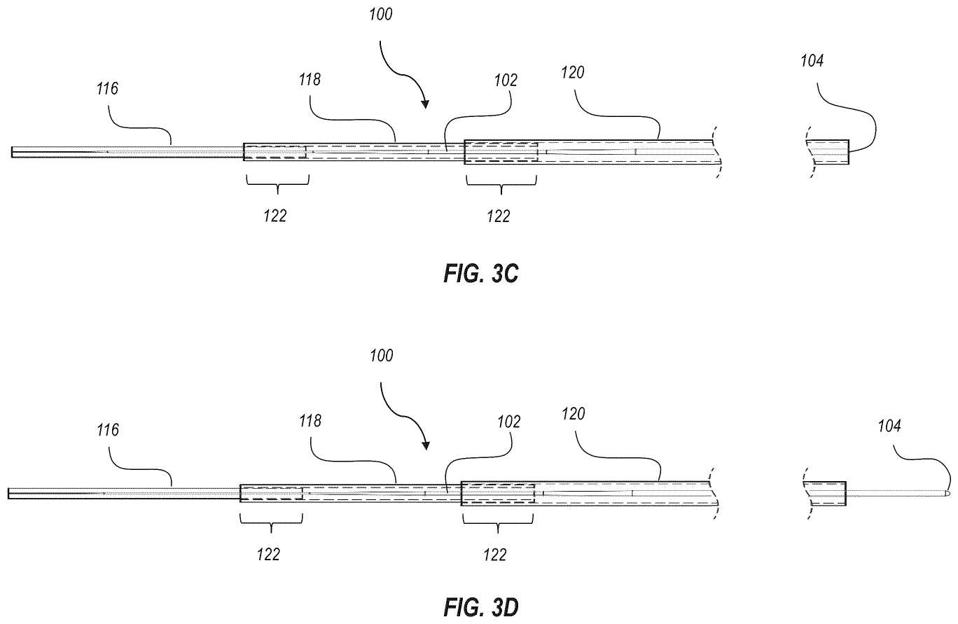

FIGS. 3A through 3D illustrate exemplary guidewire devices exhibiting relatively large outer diameters. The outer diameter of the guidewire devices can be sufficiently large to accommodate a large-bore catheter. The large diameter of the catheter is achieved through a plurality of hypotubes, each hypotube of the plurality of hypotubes containing a proximal section and a distal section as well as a proximal end and a distal end. The plurality of hypotubes is also preferably formed from a relatively flexible and elastic material such as nitinol to minimize increases in lateral bending stiffness of the device.

The plurality of hypotubes can be arranged in a nested configuration such that at least a portion of at least one hypotube passes into and is encompassed by another hypotube, except for the outermost hypotube. The plurality of hypotubes may include 2 or more hypotubes, such as 2, 3, 4, 5, 6, 7, 8, or more hypotubes.

In the illustrated embodiment, the plurality of hypotubes includes 3 hypotubes, including a first hypotube 116, a second hypotube 118, and a third hypotube 120, as shown in FIGS. 3A through 3D. In such embodiments, at least a portion of the inner member (shown as core 102 in the illustrated embodiments) is encompassed by a first hypotube 116. A proximal section of the first hypotube 116 may be encompassed by a second hypotube 118, and a proximal section of the second hypotube 118 may be encompassed by a third hypotube 120.

The outer diameter of the first hypotube 116 may be substantially similar to the inner diameter of the second hypotube 118 so as to minimize the amount of adhesive and/or other securement mechanisms needed to fix the first and second hypotubes 116, 118. Similarly, the outer diameter of the second hypotube 118 may be substantially similar to the inner diameter of the third hypotube 120. In such embodiments, the inner diameter of the first hypotube 116 is smaller than the inner diameter of the second hypotube 118, and the inner diameter of the second hypotube 118 will be smaller than the inner diameter of the third hypotube 120.

The plurality of hypotubes may be arranged such that the diameter of the guidewire 100, starting at the distal end, increases in discrete sections along the guidewire device 100 in the proximal direction. This beneficially allows for a large overall guidewire size without requiring large diameter solid sections which would significantly increase the lateral bending stiffness of the guidewire device.

This arrangement also beneficially allows for a relatively small distal tip capable of being manually bent/shaped and capable of selecting target vessels during navigation of the guidewire through the vasculature. Operators often bend or “shape” the distal tip of the guidewire because, while navigating through the patient's vasculature, it is easier to point the distal end of the guidewire toward desired vessels when the distal tip of the device includes a bent shape.

As shown, where the plurality of hypotubes includes three hypotubes, the distal end of the first hypotube 116 may be disposed distal of the distal end of the second hypotube 118 and the distal end of the second hypotube 118 may be disposed distal of the distal end of the third hypotube 120. This arrangement may also be applied to an embodiment comprising 4, 5, 6, 7, 8, or more hypotubes. A guidewire device 100 employing this arrangement could remain sufficiently flexible for intravascular navigation and be capable of accommodating a large-bore catheter.

The proximal end of each hypotube within a plurality of hypotubes may extend to an attachment point at or near the proximal end of the guidewire device 100. FIGS. 3A and 3B illustrate examples of guidewire devices 100 in which each hypotube extends from a distal attachment point to a point at or near the proximal end 104 of the device. In such a configuration, outer hypotubes may encompass more than one hypotube when the plurality of hypotubes comprise three or more hypotubes.

Alternatively, as shown in FIGS. 3C and 3D, the proximal ends of the hypotubes can terminate at different locations relative to one another, and/or a hypotube may encompass no more than one other hypotube. For example, a proximal section of each hypotube may extend along only a portion of the next largest hypotube. In such a configuration, the proximal section of each but the largest diameter hypotube is encompassed by and affixed to the distal section of the next largest hypotube, forming a joint 122. The hypotubes may be bound at the joints 122 through an adhesive and/or other suitable securement mechanism.

In some embodiments, the hypotubes within the plurality of hypotubes may exhibit a combination of the features described above, such as the proximal ends of one or more hypotubes extending from an attachment point to a point at or near the proximal end 104 of the device while the proximal sections of one or more other hypotubes do not extend all the way to the proximal end 104 of the core.

One or more hypotubes may completely encompass the proximal section of the core 102, such as shown in FIGS. 3A and 3C. For example, the proximal end of the core may be nested proximally within one or more hypotubes or may be substantially flush with a proximal end of one or more of the hypotubes, such as substantially flush with the outermost hypotube (i.e., the third hypotube 120 in the illustrated embodiment).

Alternatively, the proximal end 104 of the core 102 may extend past the proximal ends of the hypotubes. FIGS. 3B and 3D illustrate exemplary guidewires 100 wherein the proximal end 104 of the core 102 extends past the proximal ends of the hypotubes.

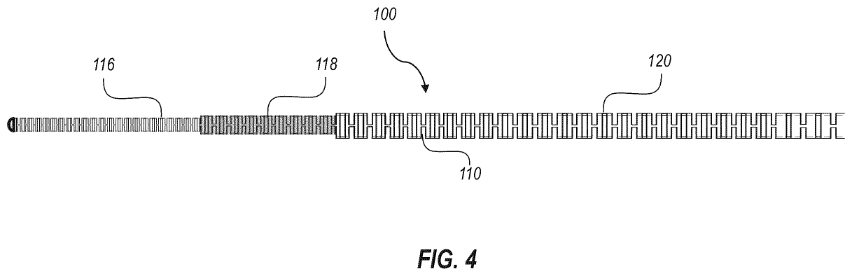

As shown in FIG. 4, one or more of the hypotubes may include fenestrations 110. FIG. 4 illustrates a guidewire device 100 with the fenestrations 110 shown cut from the plurality of hypotubes. The fenestrations 110 may be cut so as to produce a preferred bending direction or to eliminate a preferred bending direction, and may include any of the example cut patterns described above in reference to the fenestrations of FIG. 1.

The guidewire device 100 may include an outer coating such as a hydrophilic or other friction-reducing coating. The coating may encompass the full length of the guidewire device 100 or only a portion of the guidewire device 100. The coating may be applied to create a smooth outer profile of the guidewire 100. For example, the coating may be applied over the stepped shoulders formed at certain hypotube ends. The coating can beneficially form a smoother outer profile, enable more even coating deposition, and/or minimize discontinuities from one hypotube to the next. The coating may be applied via dip coating or other suitable application process.

The step size in outer diameter from the core to the first hypotube, and/or from one hypotube to the next, may range from about 0.005 inches to about 0.020 inches, such as about 0.010 inches to about 0.015 inches, or a range with endpoints selected from any two of the foregoing values. Using step sizes within the foregoing ranges beneficially provides increases in the overall outer diameter of the device without forming overly large leading shoulders (which could snag or catch vascular anatomy or an overlying catheter) at the transition points.

In one example, the first (smallest) hypotube can have an outer diameter as small as about 0.010 inches or about 0.014 inches, and the largest hypotube can have an outer diameter up to about 0.085 inches. Other embodiments may include differently sized hypotubes. For example, some embodiments may start with a first (smallest) hypotube with an outer diameter of about 0.018 inches, about 0.024 inches, or about 0.035 inches, and/or can go up to a largest hypotube that is 0.085 inches or that is less than or greater than 0.085 inches.

Relative lengths of the hypotubes can be varied. A “hypotube coverage length” is the length of the portion of the device that is coincident with any portion of a hypotube. For example, with reference to FIG. 4, the hypotube coverage length equals the length from the distal end of the first hypotube 116 to the proximal end of the third hypotube 120. This length may or may not equal the total length of the device 100, depending on whether the core 102 extends farther distally and/or proximally than the plurality of hypotubes.

Preferably, the outermost (and largest) hypotube has a length that is more than 50% of the hypotube coverage length. For example, the outermost hypotube may have a length that is 55%, 60%, 65%, 70%, 75%, 80%, 85%, 90%, or 95% of the hypotube coverage length, or a range with endpoints defined by any two of the foregoing values. This ensures minimal annular space between the catheter and the guidewire device 100 for most of the length of the device 100. In other words, this gives sufficient overall length of the device 100 with the largest outer diameter, which is beneficial for catheter tracking, while still allowing the more distal portions to taper down to smaller, more flexible outer diameters.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms.

When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device comprising:
an inner member including a proximal end and a distal end; and
a plurality of hypotubes, each hypotube of the plurality of hypotubes comprising a proximal section and a distal section,
wherein at least a portion of the inner member passes into and is encompassed by the plurality of hypotubes, and
wherein the hypotubes are arranged in a nested configuration such that, except for an outermost hypotube, a portion of each hypotube passes into and is encompassed by at least one other hypotube.

2. The intravascular device of claim 1, wherein the plurality of hypotubes comprises at least two hypotubes.

3. The intravascular device of claim 1, wherein the plurality of hypotubes comprises at least three hypotubes.

4. The intravascular device of claim 1, wherein, except for the outermost hypotube, a proximal end of each hypotube is encompassed by at least one other hypotube.

5. The intravascular device of claim 1, wherein, starting at a distal end of the device, an outer diameter of the device increases along a proximal direction at discrete points coinciding with transitions from one hypotube to another.

6. The intravascular device of claim 5, wherein the outer diameter of the device increases by 0.005 inches to 0.020 inches at each transition from one hypotube to another.

7. The intravascular device of claim 1, wherein the proximal end of the inner member is encompassed by at least one hypotube.

8. The intravascular device of claim 7, wherein the proximal end of the inner member is encompassed by each of the plurality of hypotubes.

9. The intravascular device of claim 1, wherein a proximal end of the inner member and proximal ends of one or more hypotubes are coincident at a proximal end of the intravascular device.

10. The intravascular device of claim 1, wherein the proximal end of the inner member extends farther proximally than the plurality of hypotubes.

11. The intravascular device of claim 1, further comprising a hydrophilic coating.

12. The intravascular device of claim 1, wherein one or more hypotubes of the plurality of hypotubes are formed from nitinol.

13. The intravascular device of claim 1, wherein the inner member is a solid core wire.

14. The intravascular device of claim 13, wherein the core wire is formed from stainless steel.

15. The intravascular device of claim 1, wherein one or more hypotubes of the plurality of hypotubes include a plurality of fenestrations.

16. The intravascular device of claim 15, wherein the fenestrations form an arrangement of circumferentially extending rings connected by axially extending beams.

17. The intravascular device of claim 1, wherein the plurality of hypotubes defines a hypotube coverage length, and wherein an outermost hypotube has a length that is more than 50% of the hypotube coverage length.

18. An intravascular device comprising:
an inner member including a proximal end and a distal end; and
a plurality of hypotubes comprising at least three hypotubes, each hypotube of the plurality of hypotubes comprising a proximal section and a distal section,
wherein one or more hypotubes of the plurality of hypotubes include a plurality of fenestrations,
wherein at least a portion of the inner member passes into and is encompassed by the plurality of hypotubes,
wherein the hypotubes are arranged in a nested configuration such that, except for an outermost hypotube, a portion of each hypotube passes into and is encompassed by at least one other hypotube,
wherein, starting at a distal end of the device, an outer diameter of the device increases along a proximal direction at discrete points coinciding with transitions from one hypotube to another, and
wherein the plurality of hypotubes defines a hypotube coverage length, and wherein an outermost hypotube has a length that is more than 50% of the hypotube coverage length.

19. The intravascular device of claim 18, wherein the outer diameter of the device increases by 0.005 inches to 0.020 inches at each transition from one hypotube to another.

20. The intravascular device of claim 18, wherein one or more hypotubes of the plurality of hypotubes are formed from nitinol and wherein the inner member is a solid core wire formed from stainless steel.
